(19) United States
(12) Patent Application Publication
(10) Pub. No.: US 2022/0370435 A1
(43) Pub. Date: Nov. 24, 2022

(54) Targeted therapy for treatment of chronic rhinosinusitis

(71) Applicant: ACCOLADE PHARMA LLC, Wilmington, DE (US)

(72) Inventors: Mitchell Glass, Wilmington, DE (US); Eli Meltzer, La Jolla, CA (US)

(21) Appl. No.: 17/803,326

(22) Filed: May 16, 2022

Related U.S. Application Data
(60) Provisional application No. 63/258,630, filed on May 18, 2021.

(51) Int. Cl. A61K 31/47; A61K 9/00; A61P 37/08; A61B 5/145

(52) U.S. Cl. CPC A61K 31/47; A61K 9/008; A61K 9/0075; A61P 37/08; A61B 5/14507

(57) ABSTRACT

This invention provides an effective, targeted therapy for persons suffering from an endotype of chronic rhinosinusitis which is characterized by elevated levels of LTE4 in urine.

### RELATED APPLICATION

This application claims priority under 35 U.S.C. section 119(e) from Provisional Application No. 63/258,630 filed on May 14, 2021, which is hereby incorporated in its entirety.

### FIELD OF THE INVENTION

This invention pertains to a method for providing targeted antileukotriene therapy to persons suffering from an endotype of chronic rhinosinusitis.

### BACKGROUND

Chronic rhinosinusitus (CRS) is a disease with high prevalence that can have a significant negative impact on one's health-related quality of life. It is also associated with substantial healthcare and lost-productivity costs.

CRS is defined as an inflammation of the nose and paranasal sinuses, and is associated with one or more symptoms such as: nasal blockage, nasal discharge, facial pain or pressure, and reduction or loss of sense of smell. The disease is known to be a multi-factorial disorder that may be heterogeneous in presentation and clinical course.

Given the multifaceted nature of the disease, it is not surprising that several methods of treating CRS have been developed. These methods can be broadly characterized as hygienic, pharmaceutical and surgical, and are used either separately or in combination. “Hygienic” approaches may include lavage with a saline solution. “Pharmaceutical” approaches may include the use of oral or topical corticosteroids, oral or topical antibiotics, oral cysteinyl leukotriene type-1 (CyslT1) receptor blockers, topical antifungals, topical surfactants, and subcutaneous injections of monoclonal antibodies, either separately or in combination. “Surgical” approaches are most often used when opening of the sinus ostia and/or removal of the inflammatory tissue are required, and are most effective when combined with medical therapy.

Characterization of CRS has until recently been done mostly by phenotype, principally by the presence or absence of nasal polyps. A more sophisticated approach is by endotype, i.e., classification on the basis of the underlying pathophysiological mechanism. Different phenotypes in CRS can show a very similar endotype and vice versa. This insight, together with the ability to define some endotype clusters by biomarkers of inflammation, has led to the development of new treatment options, for example, by using monoclonal antibodies for the Th2 inflammatory endotype most common in CRS with nasal polyps.

Cysteinyl leukotrienes (CysLTs, e.g., LTC4, LTD4 and LTE4) are inflammatory mediators that lead to bronchoconstriction, induction of plasma exudation and promotion of mucus secretion in the airways. It is known that LTE4 is the end-product of metabolism of CysLTs and that excretion of LTE4 in the urine provides an index of CysLT production. LTE4 has been found to be significantly higher in the urine of patients with severe asthma, compared to patients with mild-to-moderate asthma and control subjects. LTE4 elevation has also been described in conditions such as aspirin sensitivity and in forms of CRS.

Small studies of oral administration of the leukotriene modifiers, zileuton and zafirlukast, have supported a potential role for these agents in alleviating symptoms in patients with sinus symptoms and nasal polyps. However, leukotriene modifiers are not currently recommended for the treatment of CRS. “Canadian clinical practice guide-lines for acute and chronic rhinosinusitis”, by Martin Desrosiers, Gerald A Evans, Paul K Keith, Erin D Wright, Alan Kaplan, Jacques Bouchard, Anthony Ciavarel-la, Patrick W Doyle, Amin R Javer, Eric S Leith, Atreyi Mukherji, R Robert Schellenberg, Peter Small & Ian J Witterick. Allergy, Asthma & Clinical Immunology volume 7, Article number: 2 (2011)).

The failure of leukotrienes to provide more effective relief in previous studies may have been due to the use of “oral administration” of the drugs. Since oral administration of a drug inherently results in systemic exposure, there are limits to how much drug can be administered. The limit for zafirlukast is 80 mg/day, which may not be enough to be clinically effective.

There remains a need for a method to provide targeted antileukotriene therapy to a person suffering from chronic rhinosinusitis.

### SUMMARY OF THE INVENTION

One aspect of the claimed invention is a method for providing targeted antileukotriene therapy to a person suffering from chronic rhinosinusitus (CRS) comprising:
a. measuring the LTE4 level in the urine of a person suffering from CRS;
b. selecting the person for treatment if the person's LTE4 level is above 104 pg/mg Cr;
c. selecting a device that targets the high and deep nasal cavities and paranasal sinuses; and
d. treating the selected person with between about 40 micrograms/day and 1 milligram/day of zafirlukast, wherein the zafirlukast is administered topically via the device and is delivered high and deep in the nasal cavities to the regions of the osteomeatal complex and superior turbinate of the person suffering from CRS.

### DETAILED DESCRIPTION

In one aspect of the invention, the LTE4 level in the urine of a person suffering from CRS is measured on 24-hour urine specimens using the LC-MS method developed at Mayo Medical Laboratories. Many commercial diagnostic laboratories are equipped to carry out such measurements. An LTE4 level above about 104 pg/mg Cr is considered to be elevated.

In one aspect of the claimed invention, the zafirlukast is administered topically. This provides significant advantages over oral delivery of the drug. Oral delivery exposes the drug to the digestive tract, with potential for significant modification of the drug before it reaches the targeted area. Moreover, oral delivery involves systemic exposure, with risk of unwanted side effects. Appropriate delivery devices can deliver higher concentrations of a drug to the targeted tissue (the nasal passages) without systemic exposure, thereby reducing the side-effect profile and improving the efficacy of the drug.

In one aspect of the claimed invention, the zafirlukast is administered via a spray or aerosol into the nose. In one aspect of the invention, a breath-activated nasal delivery device is used to deliver the spray or aerosol past the anterior portions of the nose and into the nasal passages and cavities, preferably into the regions of the osteomeatal complex and superior turbinate. A suitable example of such a device is currently commercially available from Optinose. The device provides broad, consistent drug delivery high and deep in the nasal passages, leveraging the mucosal surfaces as the target for the treatment of CRS.

In one aspect of the invention, the zafirlukast that is administered via a breath-activated nasal delivery device is formulated as a powder, with particle size being between 10 and 18 microns. In another aspect, the zafirukast is formulated as an aqueous suspension.

1. A method for providing targeted antileukotriene therapy to a person suffering from chronic rhinosinusitus (CRS) comprising:
a. measuring the LTE4 level in the urine of a person suffering from CRS;
b. selecting the person for treatment if the person's LTE4 level is above 104 pg/mg Cr;
c. selecting a device that targets the high and deep nasal cavities and paranasal sinuses; and
d. treating the selected person with between about 40 micrograms/day and 1 milligram/day of zafirlukast, wherein the zafirlukast is administered topically via the device and is delivered high and deep in the nasal cavities to the regions of the osteomeatal complex and superior turbinate of the person suffering from CRS.

2. The method of claim 1, wherein the zafirlukast is delivered as an aqueous suspension.

3. The method of claim 1, wherein the zafirlukast is delivered as a powder.

4. The method of claim 1, wherein the zafirlukast is delivered via a breath-activated nasal delivery device.
